(19) United States
(12) Patent Application Publication
(10) Pub. No.: US 2025/0108185 A1
(43) Pub. Date: Apr. 3, 2025

(54) POSITIVE AIRWAY PRESSURE (PAP) MASKS, MASK COMPONENTS, AND MASK KITS

(71) Applicant: Sleepnet Corporation, Hampton, NH (US)

(72) Inventors: Mark Gong, Andover, MA (US); Jerry Gong, Andover, MA (US); Robert Skaff, Hampton, NH (US); Thomas Moulton, Rye, NH (US)

(21) Appl. No.: 18/889,813

(22) Filed: Sep. 19, 2024

Related U.S. Application Data
(60) Provisional application No. 63/586,020, filed on Sep. 28, 2023.

(51) Int. Cl. A61M 16/06

(52) U.S. Cl. CPC A61M 16/0622; A61M 2205/6045

(57) ABSTRACT

Positive airway pressure (PAP) masks, mask components, and mask kits are described. A mask body includes one or more tabs that insert at least partially through apertures of a mask frame, and act upon perimeter edges of the apertures to properly orient the mask body with respect to the mask frame. The one or more tabs are not configured to retain the mask frame against the mask body. The mask frame can be coupled to the mask body while the one or more tabs guide positioning of the mask body and mask frame for optimum seal between the mask body and mask frame such that they operate as an integrated unit.

### BACKGROUND

Hospital and at home patients sometimes need supplemental respiratory support. Positive pressure ventilation, in which a supply of pressurized air is delivered to the patient's airway, is often used. Positive pressure ventilation has been used to treat respiratory failure, respiratory insufficiency, and sleep apnea. There are a variety of patient interfaces which can be used to provide positive pressure ventilation including Nasal masks, Full Face masks, and Nasal Pillows for home and hospital use.

PAP, or positive airway pressure, is a treatment that uses air pressure to keep the airways open. PAP treatment typically is used by people who have breathing problems, such as sleep apnea. Sleep disorder breathing is a common disorder that causes pauses in breathing or shallow breaths while a person sleeps. As a result, not enough air reaches the person's lungs.

In obstructive sleep apnea, the person's airway collapses or is blocked during sleep. Air pressure from PAP treatment can prevent that person's airway from collapsing or becoming blocked.

PAP treatment basically involves: a PAP machine that blows therapeutic air; and a PAP mask assembly that receives the therapeutic air from the machine via an interconnecting hose. PAP mask assemblies typically comprise a mask body of rigid or pliable material (e.g., plastic) with a face-contacting cushion (e.g., a gel filled bladder) that is held in place with headgear (e.g., straps). The mask body provides the structure for: a headgear connector; straps that can be removably attached to the headgear connector; and a swiveling elbow assembly to which the hose can be attached. The cushion provides a seal against the patient's face, creating a chamber around the airway through which positive pressure ventilation can be applied.

U.S. Pat. No. 11,529,485 discloses PAP masks in which a mask body includes prongs that insert through and act upon (e.g., have a clasping or gripping effect upon) perimeter edges of apertures of a mask frame to retain the mask frame to the mask body.

### SUMMARY

A first aspect of the present disclosure relates to a positive airway pressure (PAP) mask comprising a mask body, formed from a single material, and a mask frame. The mask body comprises an outer convex surface; an inner concave surface corresponding to the outer convex surface, wherein the inner concave surface defines a chamber of the mask body; a first tab extending outwardly from the outer convex surface; and an aperture defined by a continuous perimeter extending between the outer convex surface and the inner concave surface. The mask frame comprises an arcuate portion comprising a first aperture therein, the first aperture having a continuous perimeter; and an extension element comprising an outer perimeter. The first tab of the mask body acts upon the continuous perimeter of the first aperture of the mask frame to align the mask body with respect to the mask frame. The extension element of the mask frame extends through the aperture of the mask body and the outer perimeter of the extension element friction fits to the continuous perimeter of the aperture of the mask body, thereby creating a seal between the mask frame and the mask body. The friction fit is the only mechanism of retaining the mask frame to the mask body.

In at least some embodiments of the first aspect, the first tab has a major length that extends proximate to and partially along the continuous perimeter of the first aperture of the mask body.

In at least some embodiments of the first aspect, the first tab is arcuate and extends proximate to and partially along the continuous perimeter of the first aperture of the mask body.

In at least some embodiments of the first aspect, the first tab acting upon the continuous perimeter of the first aperture of the mask frame limits rotational movement of the mask body with respect to the mask frame about a central axis extending through the extension element of the mask frame.

In at least some embodiments of the first aspect, the first tab contacts the continuous perimeter of the first aperture of the mask frame when the mask body is aligned with and friction fit to the mask frame.

In at least some embodiments of the first aspect, the portion of the continuous perimeter contacted by the first tab is arcuate.

In at least some embodiments of the first aspect, the mask body further comprises a second tab extending outwardly from the outer convex surface; the arcuate portion of the mask frame further comprises a second aperture therein, the second aperture having a continuous perimeter; and the second tab acts upon the continuous perimeter of the second aperture of the mask frame to align the mask body with the mask frame.

A second aspect of the present disclosure relates to a positive airway pressure (PAP) mask kit comprising the mask body of the first aspect of the present disclosure, and the mask frame of the first aspect of the present disclosure.

A third aspect of the present disclosure relates to a positive airway pressure (PAP) mask body formed from a single material, the PAP mask body comprising: an outer convex surface; an inner concave surface corresponding to the outer convex surface, wherein the inner concave surface defines a chamber of the PAP mask body; a first tab extending outwardly from the outer convex surface; an aperture defined by a continuous perimeter extending between the outer convex surface and the inner concave surface; a first elongated portion extending from the outer convex and inner concave surfaces, wherein the first elongated portion is configured to extend toward a first check of a user; a second elongated portion extending from the outer convex and inner concave surfaces, wherein the second elongated portion is configured to extend toward a second check of the user; and a continuous cushion attachment surface surrounding the chamber and extending along the first elongated portion and the second elongated portion, wherein the first elongated portion is configured to be folded toward the second elongated portion without causing a lower portion of the chamber to deform, wherein the first tab is configured to act upon a continuous perimeter of a first aperture of a mask frame to align the PAP mask body with respect to the mask frame, and wherein the first tab acting upon the first aperture does not retain the mask frame to the PAP mask body.

In at least some embodiments of the third aspect, the continuous perimeter of the aperture of the PAP mask body is configured to receive and friction fit to an extension element of the mask frame, thereby creating a seal between the mask frame and the PAP mask body; and the friction fit is the only mechanism of retaining the mask frame to the PAP mask body.

In at least some embodiments of the third aspect, the first tab acting upon the first aperture limits rotational movement of the PAP mask body with respect to the mask frame about a central axis extending through the extension element of the mask frame.

In at least some embodiments of the third aspect, the first tab has a major length that extends proximate to and partially along the continuous perimeter of the aperture of the PAP mask body.

In at least some embodiments of the third aspect, the first tab is arcuate and extends proximate to and partially along the continuous perimeter of the aperture of the PAP mask body.

In at least some embodiments of the third aspect, the first tab is configured to contact the continuous perimeter of the first aperture of the mask frame when the PAP mask body is aligned with and friction fit to the mask frame.

In at least some embodiments of the third aspect, the PAP mask body further comprises a second tab extending outwardly from the outer convex surface, wherein the second tab is configured to act upon a continuous perimeter of a second aperture of the mask frame to align the PAP mask body with the mask frame, and the second tab acting upon the continuous perimeter of the second aperture does not retain the mask frame to the PAP mask body.

### BRIEF DESCRIPTION OF DRAWINGS

For a more complete understanding of the present disclosure, reference is now made to the following description taken in conjunction with the accompanying drawings.

FIG. 1A is a front view of a mask body having a cushion coupled thereto.

FIG. 1B is a perspective view of the mask body and cushion of FIG. 1A.

FIG. 1C is a left-side view of the mask body and cushion of FIG. 1A.

FIG. 2A is a front view of a mask body having a cushion coupled thereto.

FIG. 2B is a perspective view of the mask body and cushion of FIG. 2A.

FIG. 2C is a left-side view of the mask body and cushion of FIG. 2A.

FIG. 3A is a front view of a mask frame.

FIG. 3B is a cross-sectional view of the mask frame of FIG. 3A taken along line A-A.

FIG. 3C is a right-side view of the mask frame of FIG. 3A.

FIG. 3D is a bottom view of the mask frame of FIG. 3A.

FIG. 4A is a front view of a mask cushion.

FIG. 4B is a bottom-planar view of the mask cushion of FIG. 4A.

FIG. 4C is a cross-sectional view of the mask cushion of FIG. 4B taken along line B-B.

FIG. 5 is a perspective view of a PAP mask include the mask body and cushion of FIGS. 1A through 1C coupled to the mask frame of FIGS. 3A through 3D.

FIG. 6 is a perspective view of a PAP mask include the mask body and cushion of FIGS. 2A through 2C coupled to the mask frame of FIGS. 3A through 3D.

### DETAILED DESCRIPTION

The present disclosure provides flexible PAP masks that include a mask body having one or more tabs that insert at least partially through apertures of a mask frame, and act upon perimeter edges of the apertures to properly orient the mask body with respect to the mask frame. The one or more tabs of the present disclosure are not configured to retain the mask frame against the mask body. That is, the one or more tabs do not act upon the perimeter edges of the apertures of the mask frame to clasp or grip the mask frame to the mask body. A mask frame of the present disclosure can be coupled to a mask body while the one or more tabs guide positioning of the mask body and mask frame for optimum seal between the mask body and mask frame such that they operate as an integrated unit.

The one or more tabs of the present disclosure provide a mechanism for a user (e.g., an aging user or one with ailments such as arthritis) to utilize an air inlet of the mask body to be the first point of registration with the mask frame while the user aligns the mask frame with the one or more tabs to properly orient the mask frame with respect to the mask body.

### Mask Bodies

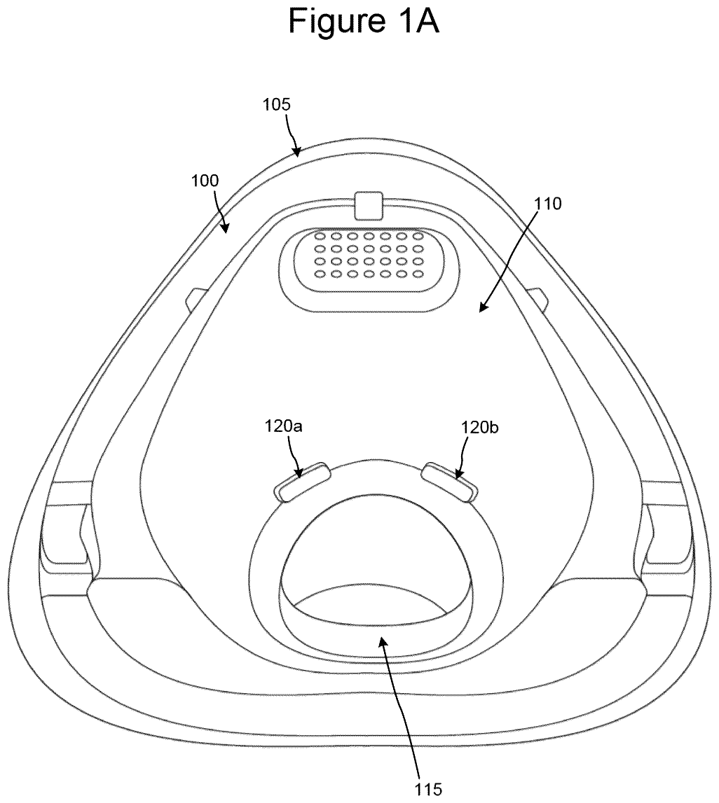

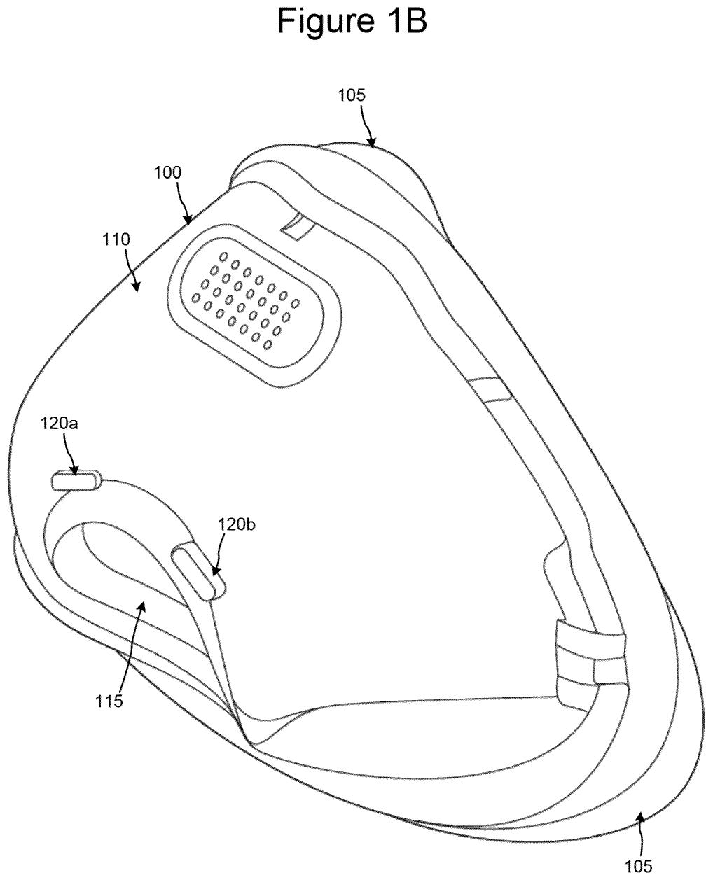

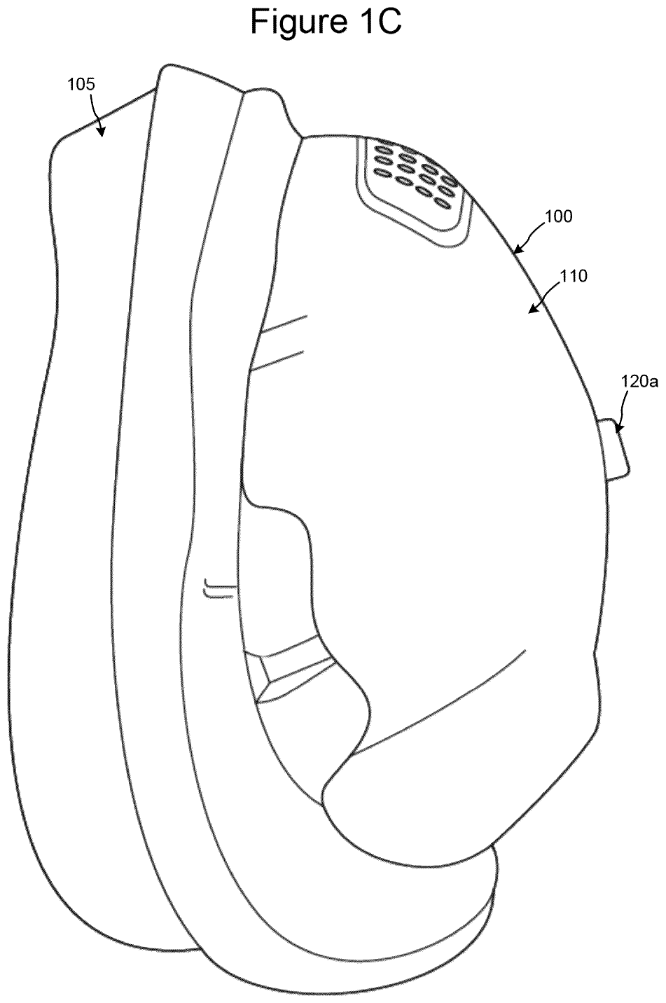

FIGS. 1A through 1C illustrate a mask body 100 having a cushion 105 coupled thereto. In some embodiments, the mask body 100 may be configured to fit over at least a portion of a nose (e.g., at least nostrils) of a user. Such may be referred to as a nasal mask body. In some other embodiments, the mask body 100 may be configured to fit over a mouth and at least a portion of a nose of a user. Such may be referred to as an oronasal mask body.

The mask body 100 includes an inner concave surface defining a chamber. The dimensions of the inner concave surface, and thus the volume of the chamber, are configurable based on nose heights of users and/or air volume requirements for proper PAP use. The mask body 100 includes an outer convex surface 110 corresponding to the inner concave surface.

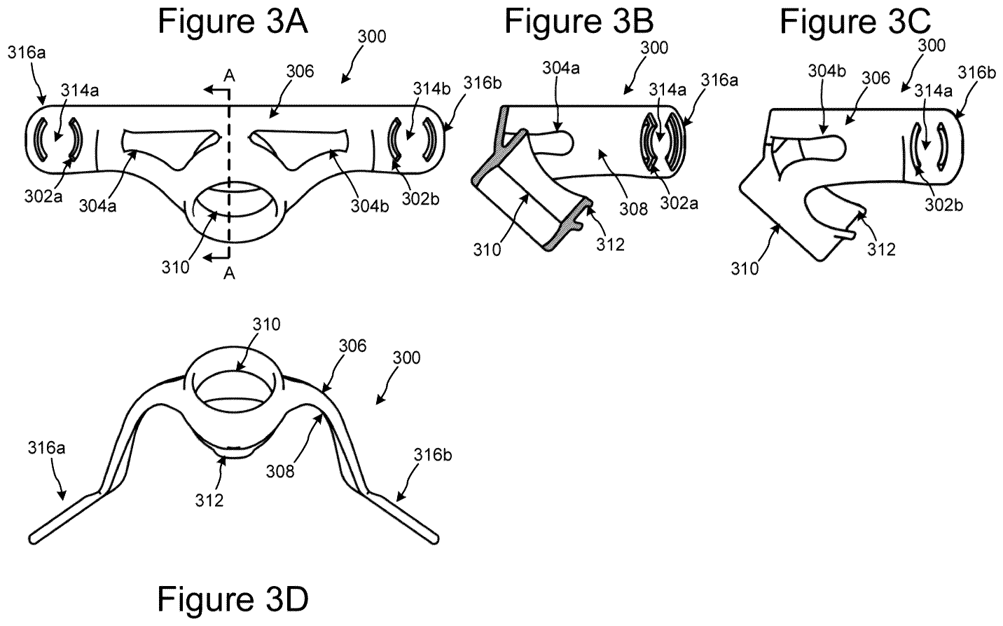

The mask body 100 includes an aperture 115 located through the outer convex surface 110 and the inner concave surface. The aperture 115 may have a continuous perimeter extending between the outer convex surface 110 and the inner concave surface, where the continuous perimeter is configured to create a seal (e.g., an airtight seal) with a portion of a mask frame 300 (described in detail herein with respect to FIGS. 3A through 3D). The size, shape, and placement of the aperture 115 is not limited to that illustrated in FIGS. 1A and 1B. The size, shape, and placement of the aperture 115 are configurable, provided that the aperture 115 may receive and create a seal with a portion of the mask frame 300.

The outer convex surface 110 may also include one or more tabs 120 extending outwardly from the outer convex surface 110. As illustrated in FIGS. 1A through IC, the outer convex surface 110 may include a first tab 120a and a second tab 120b. However, the outer convex surface 110 may include a single tab 120 or more than two tabs 120. The size, shape, and placement of the one or more tabs 120 on the outer convex surface 110 are configurable provided that the one or more tabs 120 are capable of being inserted at least partially through apertures of the mask frame 300 and act upon perimeter edges of the mask frame apertures, thereby properly orienting the mask body 100 with respect to the mask frame 300.

In some embodiments, the mask body 100 may include a retaining element (not illustrated) (e.g., a wire) configured to enable a user of the mask body 100 to form the outer convex surface 110 and the inner concave surface, and thus the chamber of the mask body 100, to features/contours of the user's face, thereby enabling a better seal of the mask body 100 to the user's face. In some embodiments, the mask body 100 may not include the retaining element. For example, in pediatric applications a sufficient seal may be provided via tension applied on the mask body 100 by headgear.

In some embodiments, the retaining element may be disposed within the mask body 100 between the outer convex surface 110 and the inner concave surface. In other embodiments, the retaining element may be affixed to the outer convex surface 110. In still other embodiments, the retaining element may be affixed to the inner concave surface.

In embodiments where the mask body 100 is configured to surround a mouth of a user, the retaining element may include a portion configured to be positioned along a chin of the user. In some embodiments, this portion of the retaining element may be stiffened, for example by molding a polycarbonate sleeve over the portion of the retaining element before the retaining element, and polycarbonate sleeve, are molded in or affixed to the mask body 100. Stiffening of this portion of the retaining element causes the portion to become more difficult to bend but not to be rigid (i.e., to be stiffened compared to the remainder of the retaining element).

It is noted that there are a number of means known to those skilled in the art to stiffen the aforementioned portion of the retaining element. A polycarbonate overmold is one of the least costly and most aesthetically attractive. It is also noted that the portion of the retaining element that is stiffened may nonetheless still have some “give” in it.

The retaining element can be constructed from any suitable material that is malleable and is capable of retaining a shape into which it is formed. In some embodiments, the retaining element may be constructed from one or more malleable metal wires or strips. In some embodiments, the retaining element may be constructed using one or more stainless steel wires or strips. In some embodiments, the retaining element may be constructed from one or more non-metal materials, such as polypropylene or some other non-metal material that is capable of retaining its shape as it is bent.

The mask body 100 additionally includes a cushion attachment surface configured to have the cushion 105 affixed thereto. Details of the cushion 105 are described herein below.

In at least some embodiments, the mask body 100 may be formed from a single material. In at least some embodiments, the mask body 100 may be formed from a single flexible material. More specifically, in at least some embodiments the inner concave surface, the outer convex surface 110, the one or more tabs 120, and the cushion attachment surface may all be formed from the same flexible material. In some embodiments, the flexible material may be a malleable plastic. In some embodiments, the malleable plastic may be polyvinyl chloride (PVC).

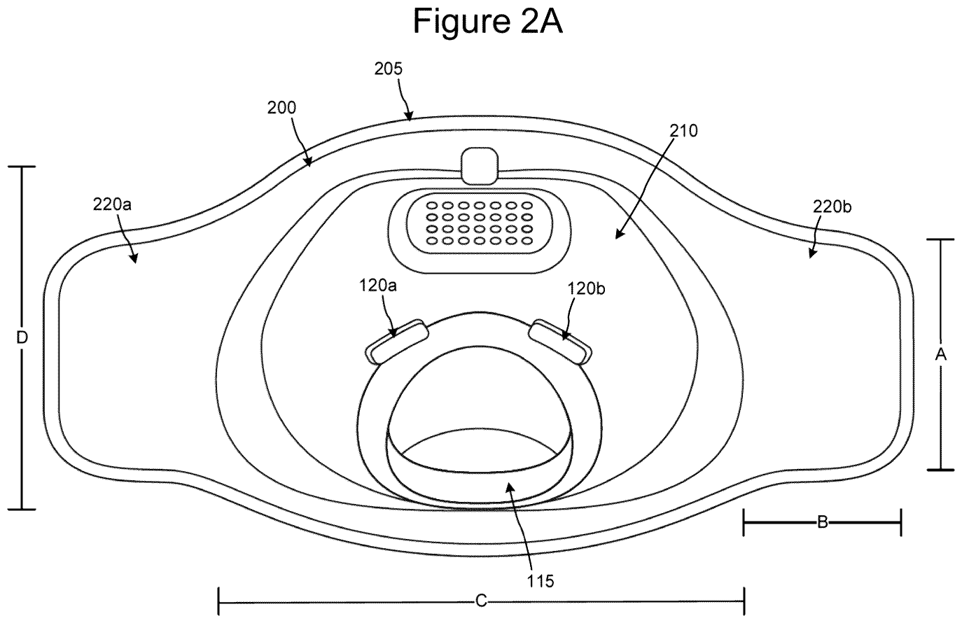

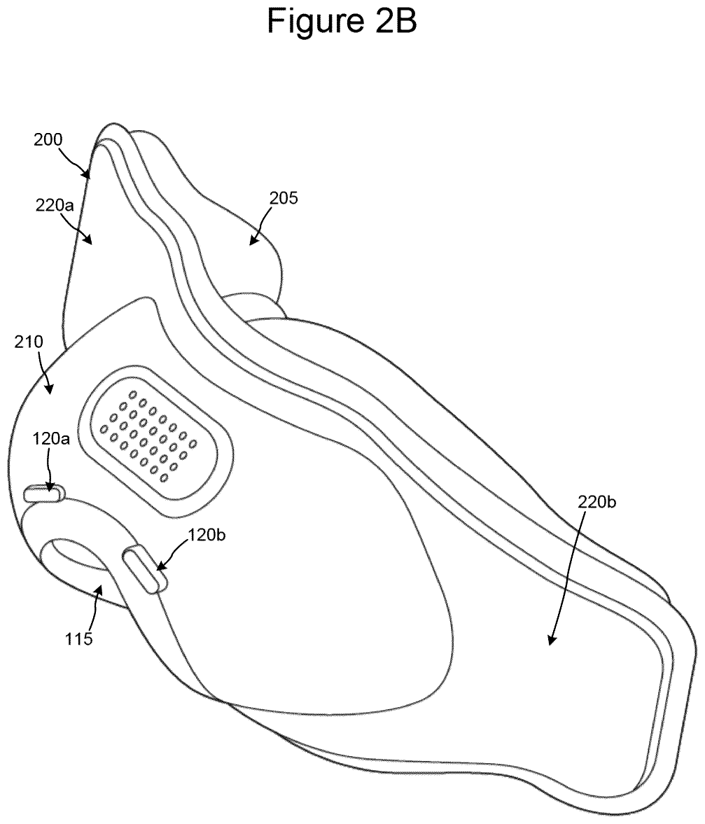

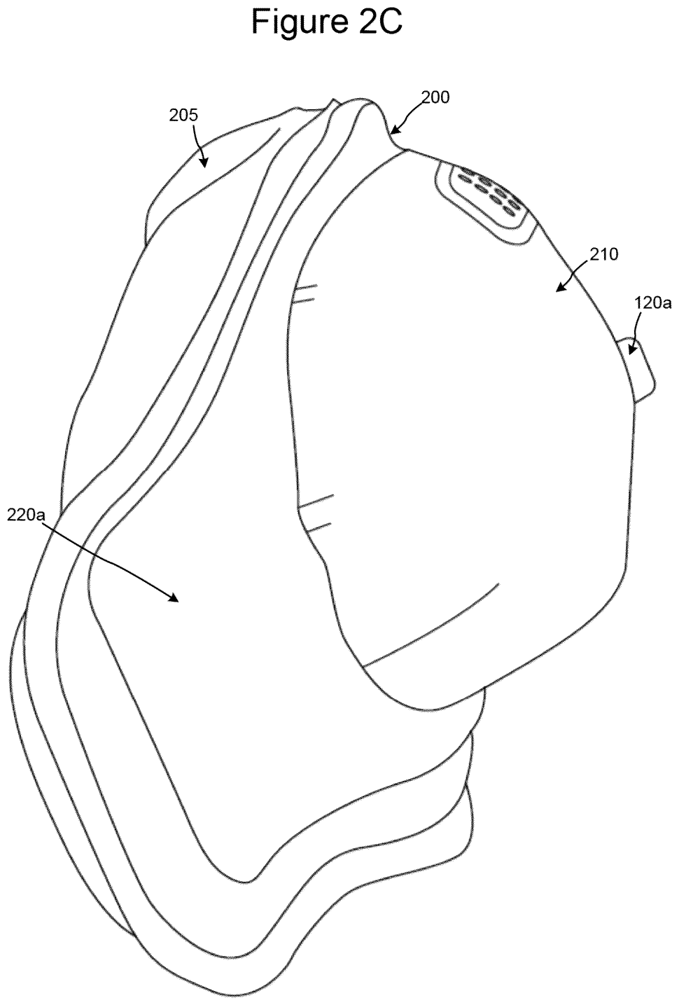

FIGS. 2A through 2C illustrate an example mask body 200 having a cushion 205 coupled thereto. In some embodiments, the mask body 200 may be a nasal mask body. In some other embodiments, the mask body 200 may be an oronasal mask body. In some embodiments, the mask body 200 may be low-profile, meaning the mask body 200 may fit over at least a portion of the user's nose, but not extend all the way up to the bridge of the user's nose. Such a mask body 200 may be more trapezoidal than triangular.

The mask body 200 includes an inner concave surface defining a chamber. The dimensions of the inner concave surface, and thus the volume of the chamber, are configurable based on nose heights of users and/or air volume requirements for proper PAP use. The mask body 200 includes an outer convex surface 210 corresponding to the inner concave surface.

The mask body 200 includes the aperture 115 located through the outer convex surface 210 and the inner concave surface. The size, shape, and placement of the aperture 115 is not limited to that illustrated in FIGS. 2A and 2B. The size, shape, and placement of the aperture 115 are configurable, provided that the aperture 115 may receive and create a seal with a portion of the mask frame 300.

The outer convex surface 210 may also include the one or more tabs 120 extending outwardly from the outer convex surface 210. As illustrated in FIGS. 2A through 2C, the outer convex surface 210 may include the first tab 120a and the second tab 120b. However, the outer convex surface 210 may include a single tab 120 or more than two tabs 120. The size, shape, and placement of the one or more tabs 120 on the outer convex surface 210 are configurable provided that the one or more tabs 120 are capable of being inserted at least partially through apertures of the mask frame 300 and act upon perimeter edges of the mask frame apertures, thereby properly orienting the mask body 200 with respect to the mask frame 300.

In some embodiments, the mask body 200 may include a retaining element (not illustrated) (e.g., a wire) configured to enable a user of the mask body 200 to form the outer convex surface 210 and the inner concave surface, and thus the chamber of the mask body 200, to features/contours of the user's face, thereby enabling a better seal of the mask body 200 to the user's face. In some embodiments, the mask body 200 may not include the retaining element. For example, in pediatric applications a sufficient seal may be provided via tension applied on the mask body 200 by headgear.

In some embodiments, the retaining element may be disposed within the mask body 200 between the outer convex surface 210 and the inner concave surface. In other embodiments, the retaining element may be affixed to the outer convex surface 210. In still other embodiments, the retaining element may be affixed to the inner concave surface.

In embodiments where the mask body 200 is configured to surround a mouth of a user, the retaining element may include a portion configured to be positioned along a chin of the user. In some embodiments, this portion of the retaining element may be stiffened, for example by molding a polycarbonate sleeve over the portion of the retaining element before the retaining element, and polycarbonate sleeve, are molded in or affixed to the mask body 200. Stiffening of this portion of the retaining element causes the portion to become more difficult to bend but not to be rigid (i.e., to be stiffened compared to the remainder of the retaining element).

It is noted that there are a number of means known to those skilled in the art to stiffen the aforementioned portion of the retaining element. A polycarbonate overmold is one of the least costly and most aesthetically attractive. It is also noted that the portion of the retaining element that is stiffened may nonetheless still have some “give” in it.

The retaining element can be constructed from any suitable material that is malleable and is capable of retaining a shape into which it is formed. In some embodiments, the retaining element may be constructed from one or more malleable metal wires or strips. In some embodiments, the retaining element may be constructed using one or more stainless steel wires or strips. In some embodiments, the retaining element may be constructed from one or more non-metal materials, such as polypropylene or some other non-metal material that is capable of retaining its shape as it is bent.

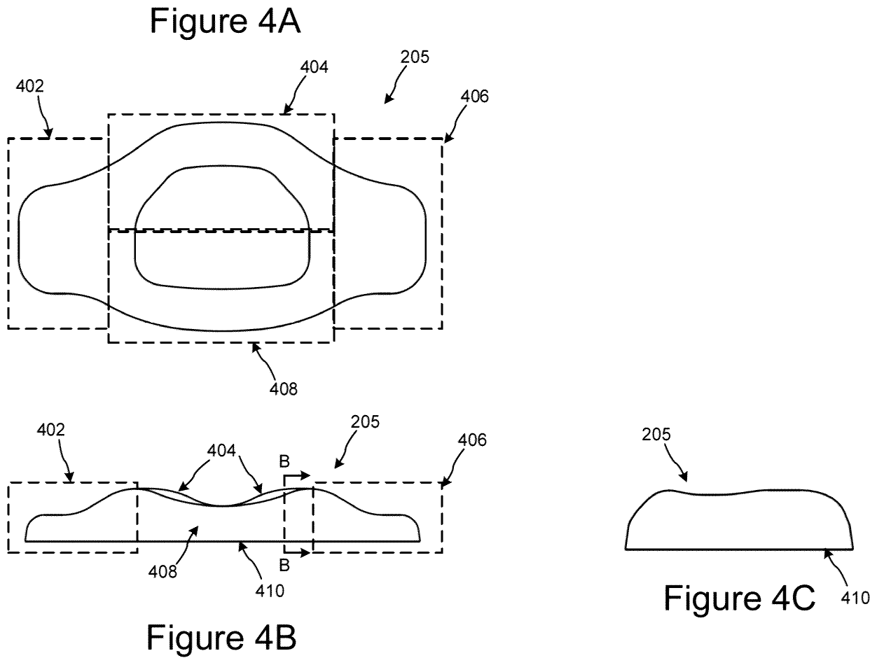

The mask body 200 may be configured with a first elongated portion 220a and a second elongated portion 220b. The first elongated portion 220a may have a width sufficient to enable the first elongated portion 220a to extend from the outer convex surface 210/chamber of the mask body 200 toward a right check of a user, and to an end of a right eye socket of the user. Likewise, the second elongated portion 220b may have a width sufficient to enable the second elongated portion 220b to extend from the outer convex surface 210/chamber of the mask body 200 toward a left check of a user, and to an end of a left eye socket of the user. By configuring the first elongated portion 220a and the second elongated portion 220b to extend to the ends of the right and left eye sockets of a user, respectively, headgear (not illustrated) of the mask may operate on and apply pressure to the first elongated portion 220a and the second elongated portion 220b to create a seal between the cushion 205 (described herein with respect to FIGS. 4A through 4C), affixed to the mask body 200, and the user's face.

The first elongated portion 220a and the second elongated portion 220b may be configured to have the same width, although the first elongated portion 220a and the second elongated portion 220b need not be configured to have the same width. In some embodiments, one or both of the first elongated portion 220a and the second elongated portion 220b may have a width B that is about one-third (⅓) to about one-half (½) of a width C of the breather chamber of the mask body 200.

Likewise, the first elongated portion 220a and the second elongated portion 220b may be configured to have the same height, although the first elongated portion 220a and the second elongated portion 220b need not be configured to have the same height. In some embodiments, one or both of the first elongated portion 220a and the second elongated portion 220b may have a height A that is less than a height D of the chamber of the mask body 200. In some embodiments, the height A may be about 50% to about 99% of the height D. In some embodiments, the height A may be about 80% of the height D. Such height configurations may be beneficial in allowing headgear of the mask to operate on and apply pressure to the first elongated portion 220a and the second elongated portion 220b to create a seal between the cushion 205 (described herein with respect to FIGS. 4A through 4C), affixed to the mask body 200, and the user's face.

The mask body 200 additionally includes a cushion attachment surface configured to have the cushion 205 affixed thereto. Details of the cushion 205 are described herein below with respect to FIGS. 4A through 4C.

In at least some embodiments, a horizontal cross-section of the mask body 200, that runs parallel to the cushion attachment surface, may be trapezoidal. The z-axis of the mask body 200, that runs from the cushion attachment surface to the inner concave surface, may be a flattened dome shape, except the base of the flattened dome is a trapezoid instead of a circle. The z-axis (e.g., height) of the mask body 200 is configured to accommodate the nose of the user and provide a chamber volume sufficient enough for fresh air (inserted into the chamber by a PAP machine) and exhaled air of the user to mix.

In at least some embodiments, the mask body 200 may be formed from a flexible material. More specifically, in at least some embodiments the inner concave surface, the outer convex surface 210, the one or more tabs 120, the first elongated portion 220a, the second elongated portion 220b, and the cushion attachment surface may all be formed from the same flexible material. In some embodiments, the flexible material may be a malleable plastic. In some embodiments, the malleable plastic may be polyvinyl chloride (PVC).

A tab 120, of the mask body 100/200, may have a major length (i.e., a dimension of the tab 120 corresponding to a longest portion of the tab 120) configured to extend proximate to and partially along the continuous perimeter of a corresponding aperture of the mask frame 300. In some embodiments, a tab 120 may be arcuate and configured to extend proximate to and partially along an arcuate portion of the continuous perimeter of a corresponding aperture of the mask frame 300. In some embodiments, the major length of a tab 120 may be arcuate.

A tab 120, of the mask body 100/200, may act upon the continuous perimeter of a corresponding aperture of the mask frame 300 and thereby limit rotational movement of the mask body 100/200 with respect to the mask frame 300 about a central axis extending through an extension element 312 (illustrated in FIGS. 3A through 3D) of the mask frame 300. When the mask body 100/200 is coupled (e.g., friction fit) to the mask frame 300, a tab 120 may contact the continuous perimeter of a corresponding aperture of the mask frame 300.

### Mask Frame

FIGS. 3A-3D illustrate an example mask frame 300 of the present disclosure. The mask frame 300 includes a convex surface 306 and a concave surface 308. In at least some embodiments, the concave surface 308 may have a concavity commensurate with a convexity of the outer convex surface 110/210 of the mask body 100/200.

The mask frame 300 may include one or more apertures 304 within and extending between the convex surface 306 and the concave surface 308. As illustrated, the mask frame 300 may include a first aperture 304a and a second aperture 304b). However, the mask frame 300 may include a single aperture 304 or more than two apertures 304. The first aperture 304a may be configured to receive the first tab 120a and the second aperture 304b may be configured to receive the second tab 120b. The first tab 120a and the second tab 120b may act upon the edge perimeters of the first aperture 304a and the second aperture 304b, respectively, to align but not retain the mask frame 300 with respect to the mask body 100/200.

In some embodiments, an aperture 304 of the mask frame 300 may be elongate, in the form of a slot, as illustrated in FIGS. 3A-3D. However, the size, shape, and placement of an aperture 304 in the mask frame 300 is not limited thereto. It is noted that the size, shape, and placement of an aperture 304 are configurable, provided that the aperture 304 is configured to receive a tab 120 of the mask body 100/200 and be acted upon to align but not retain the mask frame 300 with respect to the mask body 100/200. For example, in some embodiments an aperture 304 may be shaped to be commensurate with a shape of a tab 120. For example, a tab 120 is cylindrical, the aperture 304 may be circular.

In some embodiments, instead of being configured with one or more apertures 304, the mask frame 300 may include one or more indentations in the concave surface 308 of the mask frame 300, and optionally corresponding one or more protrusions extending from the convex surface 306. In such embodiments, the one or more indentations may be configured to receive one or more tabs 120, respectively, to align but not retain the mask frame 300 with respect to the mask body 100/200.

The mask frame 300 includes a first end portion 316a and a second end portion 316b. As illustrated in FIG. 3D, the first end portion 316a and the second end portion 316b may be angularly offset from the convex surface 306.

In at least some embodiments, one or both of the first end portion 316a and the second end portion 316b may have a magnet 314 located therein or affixed thereto. For example, the first end portion 316a may have a first magnet 314a located therein or affixed thereto and/or the second end portion 316b may have a second magnet 314b located therein or affixed thereto. The first magnet 314a and the second magnet 314b may be configured to attract corresponding magnets of headgear, such that a user can locate where the headgear is to attach to the mask frame 300 (e.g., if the user needs to disconnect the headgear from the mask frame 300 in the middle of the night and thereafter reattach same).

The size, shape, and placement of the first magnet 314a in or on the first end portion 316a are configurable, provided that the first magnet 314a is capable of interacting with (i.e., through attractive magnetic forces) a corresponding magnet of headgear. Likewise, the size, shape, and placement of the second magnet 314b in or on the second end portion 316b are configurable, provided that the second magnet 314b is capable of interacting with (i.e., through attractive magnetic forces) a corresponding magnet of headgear.

The mask frame 300 may be configured to connect to headgear for holding the mask body 100/200 against the face of a user. In at least some embodiments, the first end portion 316a may include a first headgear attachment portion 302a (represented by the two arcuate apertures in FIGS. 3A-3B), and the second end portion 316b may include a second headgear attachment portion 302b (represented by the two arcuate apertures in FIGS. 3A and 3C).

It is noted that there are various art-known/industry-known headgear, any of which may be used with the PAP mask of the present disclosure. It thus follows that the first headgear attachment portion 302a and the second headgear attachment portion 302b may be configured to connect to one or more art-known/industry-known headgear. Notwithstanding, the first headgear attachment portion 302a and the second headgear attachment portion 302b may be configured to connected to proprietary headgear, in at least some embodiments.

The mask frame 300 is configured to receive and connect to a tube configured to provide pressurized air. In at least some embodiments, the mask frame 300 may include a ball and socket connector 310 configured to receive and provide a seal (e.g., an airtight seal) with the tube. The ball and socket connector 310 is a coupling mechanism that includes a spherical socket configured to receive a corresponding spherical end of the tube. In some embodiments, the end of the tube may be semi-spherical but nonetheless capable of providing a seal (e.g., an airtight seal) with the walls of the spherical socket of the ball and socket connector 310. A spherical end of a tube and a semi-spherical end of a tube may each be referred to as a ball portion of a tube herein. The spherical nature of the ball and socket connector 310, combined with the spherical (or semi-spherical) end of the tube, allows for multidirectional movement and rotation of the tube within the ball and socket connector 310. Such multidirectional movement permits a user to sleep at different positions with respect to the compressor/motor of the PAP without experiencing tube drag, or with experiencing less tube drag than other known PAP configurations.

In at least some embodiments, the mask frame 300 may include an extension element 312 that extends from the end of the ball and socket connector 310 corresponding to the concave surface 308 of the mask frame 300. The extension element 312 may be configured to be inserted through the aperture 115 of the mask body 100/200 when the mask body 100/200 is connected to the mask frame 300. In at least some embodiments, a seal between the mask frame 300 (and more particularly the tube connected to the ball and socket connector 310) and the chamber of the mask body 100/200 may be provided solely by the extension element 312 having an outer perimeter commensurate with a perimeter wall of the aperture 115 of the mask body 100/200. In such embodiments, the size and shape of the outer perimeter, of the extension element 312, and the perimeter wall of the aperture 115 are configurable provided that they are sufficiently similar to provide a seal (e.g., an airtight seal).

The mask frame 300 may be constructed using a thermoplastic polymer. In some embodiments, the thermoplastic polymer may be polypropylene. The mask frame 300 may additionally or alternatively be constructed using one or more rather flexible materials, such as PVC and/or acrylonitrile-butadiene-styrene (ABS). The mask frame 300 may additionally or alternative be construed using one or more malleable metals, which may bend under applied force and retain form adjustments.

### Mask Cushions

The cushion 105/205 may be removably or permanently coupled to the cushion attachment surface of the mask body 100/200. In some embodiments, the cushion 105/205 may be permanently adhered (e.g., using glue or another adhesive) to the cushion attachment surface.

The cushion 105/205 can be comprised of one or more formed flexible materials having a durometer value of 60 or less on a Type 000 Shore scale, a durometer value of 50 or less on a Type 000 Shore scale, a durometer value of 40 or less on a Type 000 Shore scale, a durometer value of 30 or less on a Type 000 Shore scale, a durometer value of 20 or less on a Type 000 Shore scale, or a durometer value of 10 or less on a Type 000 Shore scale.

In some embodiments, the cushion 105/205 can be comprised of one or more formed flexible materials having a durometer value of about 60 to about 90 on Type 000 Shore scale. In some embodiments, the formed flexible materials may have a durometer value of about 60, about 65, about 70, about 75, about 80, about 85, or about 90 on the Type 000 Shore scale.

Example cushions of the present disclosure are silicone gel bladders, air-filled bladders, foam-filled bladders, liquid rubber-filled bladders, molded foam and molded rubber.

The Shore durometer is a device known to one of ordinary skill in the art for measuring the hardness of a material, typically of polymers, elastomers, and rubbers. Higher numbers on the scale indicate a greater resistance to indentation and thus harder materials. Lower numbers indicate less resistance and softer materials. The term is also used to describe a material's rating on the scale, as in an object having a “Shore durometer of 90.” Shore durometers are designed to measure specific scale ranges. A Type 000 durometer measures the hardness of very soft materials.

By configuring the cushion 105/205 to have a durometer value of 60 or less on a Type 000 Shore scale, the mask body 100/200 may contour to the user's face as it experiences tension from headgear, and the cushion 105/205 may fill in the gaps not accommodated by pure contouring of the mask body 100/200. The cushion 105/205 does this while maintaining a thin profile and reasonable comfort for the user.

The cushion 105/205 may include an external membrane bladder containing a fluid, for example a gel, for example a silicone gel. The external membrane bladder may be formed from one or more thermopolymer sheets. Thermopolymer sheet material is known to one of ordinary skill in the art. Examples of thermopolymer sheet materials include those comprising urethane, polyurethane, latex, nitrile, and other natural and synthetic polymers. The external membrane bladder may be formed from a sheet of thermopolymer comprising, for example, first and second thermopolymer sheet portions. The term “thermopolymer sheet portion” is defined to mean a portion of a single sheet folded or otherwise manipulated to form more than one face of the external membrane bladder.

In some embodiments, gel (e.g., silicone gel) may be used to fill the external membrane bladder. According to the present disclosure, a cushion 105/205, having an external membrane bladder filled with gel, may have a durometer value of 60 or less on a Type 000 Shore scale, a durometer value of 50 or less on a Type 000 Shore scale, a durometer value of 40 or less on a Type 000 Shore scale, a durometer value of 30 or less on a Type 000 Shore scale, a durometer value of 20 or less on a Type 000 Shore scale, or a durometer value of 10 or less on a Type 000 Shore scale. However, it is noted that the present disclosure envisions materials, other than gel, being used to fill the external membrane bladder while still maintaining a durometer value of 60 or less on a Type 000 Shore scale.

The cushion 105 may be triangular in cross-section with three primary external faces. The cushion 105 may include a skin contact face, an attachment face attached to the attachment surface of the mask body 100, and a third face. In cross-section, the skin contact face may be the longest face. The attachment face can be longer than, shorter than or equal in length to the third face. In some embodiments, the third face has a length less than about 70% to 90% of the length of the skin contact face. The angle formed between the attachment face and the third face may be between 70 degrees and 90 degrees.

The third face may occupy the inner perimeter of the cushion 105 and the skin contact face may occupy the outer perimeter of the cushion 105. In cross-section, the transition between the faces of the cushion 105, particularly the transition between the third face and the skin contact face, may appear rounded, slightly rounded, or even flat.

In some embodiments, the cushion 105 can have more than three primary faces. One skilled in the art will recognize, for example, that a trapezoidal seal will offer many of the advantages described herein in connection with a triangular cushion, and one skilled in the art, guided by the present disclosure, can make and use such trapezoidal seals using no more than routine experimentation.

In some embodiments, the cushion 105 is formed by drawing a first thermopolymer sheet portion into a molding apparatus with heat and vacuum to form the skin contact face and the third face. A molding apparatus is, in its simplest form, a vacuum mold having a cavity or well in the shape of the skin contact face and third face. In a first step, a polymer sheet may be heated and drawn under vacuum to form the exterior bladder boundary of the skin contact face and the third face. The vacuum may be created through the use of air holes in the bottom of the mold cavity. Because the skin contact face and the third face meet at the bottom of the heated vacuum cavity, the transition between those faces may be rounded, thereby enabling removal of the molded product and generally facilitating the process. A gel, foam, liquid rubber or other like viscous material may then be poured into the mold to fill the cavity formed by the first polymer sheet. A second polymer sheet (or another portion of the same polymer sheet) may then be overlaid on the viscous material-filled cavity and the edges of the two polymer sheets may be heat-bonded to seal the viscous material within the cushion 105.

When drawn under heat and vacuum, the drawn thermopolymer sheet portion(s) will be made thinner than the starting stock material. One of skill in the art will recognize the effects of vacuum drawing on thermopolymer sheet thickness. For particular applications, one of skill in the art may select polymer sheets having varying thicknesses. For example, a thinner film will maximize the ability for the filled cushion to contour. Thicker films are often used for deeper draws in the heated mold.

In some embodiments, the cushion 105 may be configured with one or more local lobes positioned at locations correspond with areas of dramatic feature change in the contour of a face to be fit (e.g., around the nose and the bridge of the nose). To enable inclusion of the one or more local lobes, the cavity of the molding apparatus may contain a local cavity (i.e., a cavity deeper than the cavity forming adjacent non-lobe portions) deepening the draw to form the one or more local lobes. A local lobe may have an upper portion and a lower portion. The upper portion may be both local and prominent, rising dramatically from the non-lobe portion of the cushion 105. The upper portion may be trapezoidal in shape based on the manufacturing process.

The side projection of a local lobe can be broken down into a generally rectangular, lower portion, the height of which may approximate the height of side projections of adjacent non-lobe portions of the cushion 105, and a generally trapezoidal portion sitting atop the rectangular portion. The trapezoidal portion represents the local lobe rising dramatically from the adjacent non-lobe portion of the cushion 105. The angled side legs of the trapezoid may form an average angle with the top of the rectangular portion and the average angle may be between 30 degrees and 89 degrees.

In designing the local lobes, one of skill in the art will recognize and consider several design factors. The lobe should be large enough to seal around features of the face with dramatic structural changes. An example given previously is the nose and the bridge of the nose. It will also be recognized that lobes that are too large will also result in a “leaky” seal caused by the upper portion of the local lobe lifting the adjacent non-lobe portion off the face.

The cushion 105 may comprise one or more local lobes extending the skin contact face and the third face for sealing areas of substantial variation in facial structure of the user. For example, on a mask that covers the nose and mouth, local lobes may be located at the portion of the cushion 105 that positions on either side of the nose and the bridge of the nose to compensate for the extreme change in contour of the face at this position. Likewise, local lobes may be advantageous on the cushion 105 when the mask is a nasal mask or one covering the eyes, as well as the nose and mouth, as areas of substantial variation in facial structure can vary depending on the type of mask used. For example, when the mask body 100 is that of a nasal mask, local lobes may be beneficial on either side of the nose and the bridge of the nose. Moreover, when the mask body 100 is that of a mask covering the eyes, local lobes may be beneficially located adjacent to the eye sockets, under the lower lip or at the checks.

The cushion 105, when configured with the three primary external faces, is designed to form a seal between the skin contact face and the face of the user. As contact is made, the third face bulges and the skin contact face “lays down” increasing the contact area with the face. The triangular shape of the seal of the present disclosure allows for the skin contact face to “lay down” (or redirect) allowing the majority of the surface of the plane to make contact and remain in contact with the user's face. As discussed above, the modified prior art semi-circular sealing element of the prior art forms an inferior seal.

Examples of the cushion 105 are those described in U.S. Pat. No. 10,974,007, the contents of which are hereby incorporated by reference in their entirety.

FIGS. 4A-4C illustrate an example of the cushion 205 of the mask body 200. The cushion 205 may be viewed as including four portions: a first elongated portion 402, an upper portion 404, a second elongated portion 406, and a lower portion 408. The first elongated portion 402 may abut the second elongated portion 220b, of the mask body 200, when an attachment face 410, of the cushion 205, is permanently or removably affixed to the cushion attachment surface of the mask body 200. Likewise, the second elongated portion 406 may abut the first elongated portion 220a, of the mask body 200, when the attachment face 410, of the cushion 205, is permanently or removably affixed to the cushion attachment surface of the mask body 200.

In at least some embodiments, the attachment face 410 may be glued to the cushion attachment surface (e.g., using any commercially available or proprietary adhesive). In at least some embodiments, the cushion attachment surface, of the mask body 200, may include one or more protrusions (or protruding edges) configured to be inserted within the cushion 205 through the attachment face 410, thereby physically and removably coupling the cushion 205 to the mask body 200. It thus follows that the cushion 205 is replaceable.

As illustrated in FIG. 4A, the upper portion 404 and the lower portion 408 may each be positioned between the first elongated portion 402 and the second elongated portion 406. The upper portion 404 is configured to contact and create a seal with a nose of a user. When the cushion 205 is configured for affixation to a nasal mask body, the lower portion 408 may be configured to contact and create a seal with an upper lip of a user. When the cushion 205 is configured for affixation to an oronasal mask body, the lower portion 408 may be configured to contact and create a seal with a chin of a user.

In at least some embodiments, the upper portion 404 and the lower portion 408 may each be configured with at least three primary faces: a skin contact face; the attachment face 410; and a third face. The skin contact face may be the longest face of the faces. In at least some embodiments, the third face may have a length that is less than about 70% to about 90% of the length of the skin contact face. In instances where the upper portion 404 and/or the lower portion 408 are triangular in cross-section, the angle formed between the attachment face 410 and the third face may be between about 70° and about 90°. In at least some embodiments (e.g., for manufacturing reasons), a range of between about 70° and 89° degrees may be preferred.

The third face may occupy an inner perimeter of the cushion 205 and the skin contact face may occupy the outer perimeter of the cushion 205. In cross-section, the transition between the faces, particularly the transition between the third face and the skin contact face, may be rounded, slightly rounded, or even flat. The reason for the rounded or flat nature of the transition may be results of the manufacturing process used to create the cushion 205.

In at least some embodiments, the upper portion 404 and/or the lower portion 408 may have more than three primary faces. One skilled in the art will recognize, for example, that a trapezoidal cross-section may be beneficial in at least some instances.

In at least some embodiments, the cushion 205 may include an external membrane bladder containing a fluid or gel. The external membrane bladder may be formed from one or more thermopolymer sheets. Thermopolymer sheet material is known to one of ordinary skill in the art. Examples of such materials include sheets comprising urethane, polyurethane, latex, nitrile, and other natural and synthetic polymers. The external membrane bladder may be formed from a sheet of thermopolymer comprising, for example, a first and a second thermopolymer sheet portion. The term “thermopolymer sheet portion” is defined to mean a portion of a single sheet folded or otherwise manipulated to form more than one face of the cushion 205.

In some embodiments, silicone gel may be used to fill the external membrane bladder. According to the present disclosure, a mask cushion, having an external membrane bladder filled with silicone gel may have a durometer value of 60 or less on a Type 000 Shore scale; a durometer value of 50 or less on a Type 000 Shore scale; a durometer value of 40 or less on a Type 000 Shore scale; a durometer value of 30 or less on a Type 000 Shore scale; a durometer value of 20 or less on a Type 000 Shore scale; or a durometer value of 10 or less on a Type 000 Shore scale. However, it is noted that the present disclosure envisions materials other than silicone gel may be used to fill the external membrane bladder while still maintaining a durometer value of 60 or less on a Type 000 Shore scale.

In some embodiments, the cushion 205 may be configured with a filled external membrane bladder. For example, the cushion 205 may be comprised of foam and/or rubber (e.g., liquid silicone rubber) that is molded or otherwise shaped (e.g., by cutting) without the need for an external membrane bladder.

### Mask Kits

The present disclosure provides various mask kits.

In some embodiments, a mask kit may include the mask body 100 having the cushion 105 attached thereto and the mask frame 300 decoupled from the mask body 100.

In some embodiments, a mask kit may include the mask body 100 having the cushion 105 attached thereto as well as the mask frame 300 coupled to the mask body 100.

In some embodiments, a mask kit may include the mask body 200 having the cushion 205 attached thereto and the mask frame 300 decoupled from the mask body 200.

In some embodiments, a mask kit may include the mask body 200 having the cushion 205 attached thereto as well as the mask frame 300 coupled to the mask body 100.

In some embodiments, a mask kit may include the mask body 100 having the cushion 105 attached thereto, the mask body 200 having the cushion 205 attached thereto, and the mask frame 300 decoupled from the mask bodies 100/200.

In some embodiments, a mask kit may include the mask body 100 having the cushion 105 attached thereto, the mask body 200 having the cushion 205 attached thereto, and the mask frame 300 coupled to one of the mask bodies 100/200.

### PAP Masks

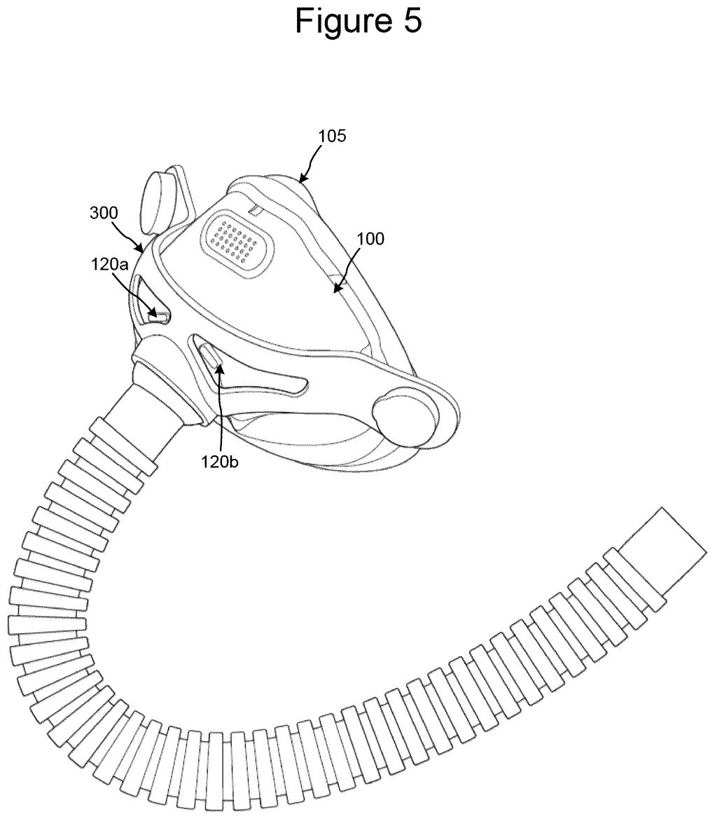

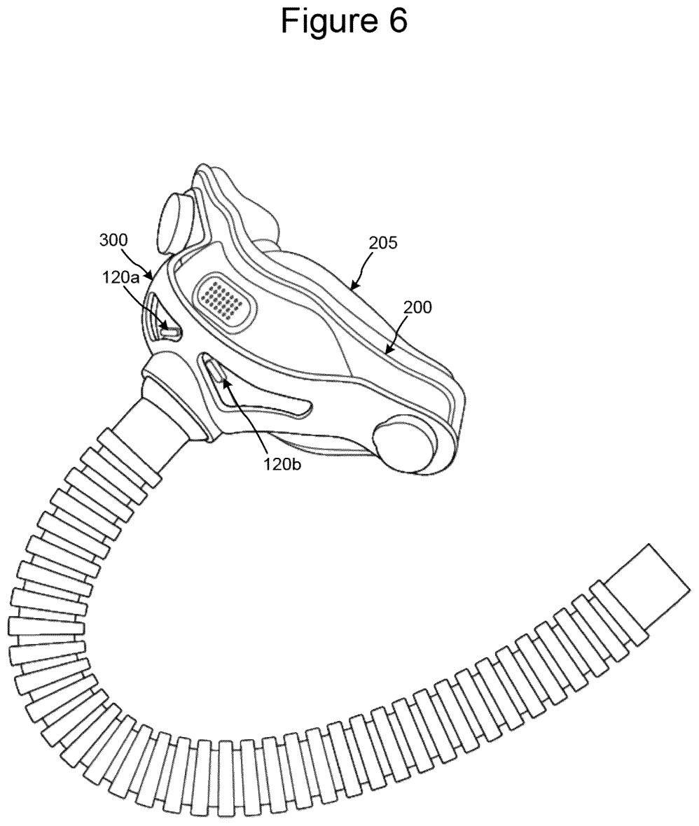

FIG. 5 illustrates a PAP mask include the mask body 100 coupled to the mask frame 300. FIG. 6 illustrates a PAP mask include the mask body 200 coupled to the mask frame 300. In each of the foregoing PAP masks, the mask body 100/200 may be retained against the mask frame 300 solely by the extension element 312 of the mask frame 300 being positioned within the aperture 115 of the mask body 100/200, and the continuous perimeter of the aperture 115 friction fitting against the extension element 312. This friction fitting of the may be the only mechanism by which the mask body 100/200 is retained against the mask frame 300. As illustrated in FIGS. 5 and 6, the first tab 120a and the second tab 120b do not function to retain the mask body 100/200 against the mask frame 300. Rather, the first tab 120a and the second tab 120b are configured to simply align the mask body 100/200 with respect to the mask frame 300 about a central axis extending through the extension element 312 of the mask frame 300.

To assembly the PAP mask, the user may partially insert the extension element 312 of the mask frame 300 into the aperture 115 of the mask body 100/200. Then, the user may rotate the mask frame 300 with respect to the mask body 100/200, about the central axis extending through the extension element 312, until the one or more tabs 120 of the mask body 100/200 align with corresponding apertures (and more specifically continuous perimeters of the apertures) of the mask frame 300. Once aligned, the user may finish inserting the extension element 312 into the aperture 115 of the mask body 100/200 until the outer convex surface 110/210 of the mask body 100/200 is abutting or proximate the concave surface 308 of the mask frame 300.

While the present invention has been particularly described in conjunction with specific embodiments, it is evident that many alternatives, modifications, and variations will be apparent to those skilled in the art in light of the foregoing description. It is therefore contemplated that the appended claims will embrace any such alternatives, modifications, and variations as falling within the true scope and spirit of the present invention.

What is claimed is:

1. A positive airway pressure (PAP) mask comprising:
a mask body formed from a single material, the mask body comprising:
an outer convex surface;
an inner concave surface corresponding to the outer convex surface, wherein the inner concave surface defines a chamber of the mask body;
a first tab extending outwardly from the outer convex surface; and
an aperture defined by a continuous perimeter extending between the outer convex surface and the inner concave surface; and
a mask frame comprising:
an arcuate portion comprising a first aperture therein, the first aperture having a continuous perimeter, and
an extension element comprising an outer perimeter,
wherein the first tab of the mask body acts upon the continuous perimeter of the first aperture of the mask frame to align the mask body with respect to the mask frame,
wherein the extension element of the mask frame extends through the aperture of the mask body and the outer perimeter of the extension element friction fits to the continuous perimeter of the aperture of the mask body, thereby creating a seal between the mask frame and the mask body, and
wherein the friction fit is the only mechanism of retaining the mask frame to the mask body.

2. The PAP mask of claim 1, wherein the first tab has a major length that extends proximate to and partially along the continuous perimeter of the first aperture of the mask body.

3. The PAP mask of claim 1, wherein the first tab is arcuate and extends proximate to and partially along the continuous perimeter of the first aperture of the mask body.

4. The PAP mask of claim 1, wherein the first tab acting upon the continuous perimeter of the first aperture of the mask frame limits rotational movement of the mask body with respect to the mask frame about a central axis extending through the extension element of the mask frame.

5. The PAP mask of claim 1, wherein the first tab contacts the continuous perimeter of the first aperture of the mask frame when the mask body is aligned with and friction fit to the mask frame.

6. The PAP mask of claim 5, wherein the portion of the continuous perimeter contacted by the first tab is arcuate.

7. The PAP mask of claim 1, wherein:
the mask body further comprises a second tab extending outwardly from the outer convex surface;
the arcuate portion of the mask frame further comprises a second aperture therein, the second aperture having a continuous perimeter; and
the second tab acts upon the continuous perimeter of the second aperture of the mask frame to align the mask body with the mask frame.

8. A positive airway pressure (PAP) mask kit comprising:
the mask body of claim 1; and
the mask frame of claim 1.

9. A positive airway pressure (PAP) mask body formed from a single material, the PAP mask body comprising:
an outer convex surface;
an inner concave surface corresponding to the outer convex surface, wherein the inner concave surface defines a chamber of the PAP mask body;
a first tab extending outwardly from the outer convex surface;
an aperture defined by a continuous perimeter extending between the outer convex surface and the inner concave surface;
a first elongated portion extending from the outer convex and inner concave surfaces, wherein the first elongated portion is configured to extend toward a first cheek of a user;
a second elongated portion extending from the outer convex and inner concave surfaces, wherein the second elongated portion is configured to extend toward a second cheek of the user; and
a continuous cushion attachment surface surrounding the chamber and extending along the first elongated portion and the second elongated portion,
wherein the first elongated portion is configured to be folded toward the second elongated portion without causing a lower portion of the chamber to deform,
wherein the first tab is configured to act upon a continuous perimeter of a first aperture of a mask frame to align the PAP mask body with respect to the mask frame, and
wherein the first tab acting upon the first aperture does not retain the mask frame to the PAP mask body.

10. The PAP mask body of claim 9, wherein:
the continuous perimeter of the aperture of the PAP mask body is configured to receive and friction fit to an extension element of the mask frame, thereby creating a seal between the mask frame and the PAP mask body; and
the friction fit is the only mechanism of retaining the mask frame to the PAP mask body.

11. The PAP mask body of claim 10, wherein the first tab acting upon the first aperture limits rotational movement of the PAP mask body with respect to the mask frame about a central axis extending through the extension element of the mask frame.

12. The PAP mask body of any one of claims 9, wherein the first tab has a major length that extends proximate to and partially along the continuous perimeter of the aperture of the PAP mask body.

13. The PAP mask body of claim 9, wherein the first tab is arcuate and extends proximate to and partially along the continuous perimeter of the aperture of the PAP mask body.

14. The PAP mask body of claim 9, wherein the first tab is configured to contact the continuous perimeter of the first aperture of the mask frame when the PAP mask body is aligned with and friction fit to the mask frame.

15. The PAP mask body of claim 9, further comprising a second tab extending outwardly from the outer convex surface, wherein the second tab is configured to act upon a continuous perimeter of a second aperture of the mask frame to align the PAP mask body with the mask frame, and the second tab acting upon the continuous perimeter of the second aperture does not retain the mask frame to the PAP mask body.
